(19) United States
(12) Patent Application Publication
(10) Pub. No.: US 2025/0090239 A1
(43) Pub. Date: Mar. 20, 2025

(54) METHOD OF REGISTERING A PATIENT WITH MEDICAL INSTRUMENT NAVIGATION SYSTEM

(71) Applicants: Acclarent, Inc., Irvine, CA (US); Biosense Webster (Israel) Ltd., Yokneam (IL)

(72) Inventors: George Gusein, Acre (IL); Cesar Fuentes-Ortega, Pasadena, CA (US); Kobi Koren, Haifa (IL); Christopher A. Schutt, Bozeman, MT (US)

(21) Appl. No.: 18/883,237

(22) Filed: Sep. 12, 2024

Related U.S. Application Data
(60) Provisional application No. 63/539,345, filed on Sep. 20, 2023.

(51) Int. Cl. A61B 34/20; A61B 34/10

(52) U.S. Cl. CPC A61B 34/20; A61B 34/10; A61B 2034/105; A61B 2034/2059; A61B 2034/2065

(57) ABSTRACT

Position data is captured for a first plurality of registration points along an anterior region of a head of a patient, based on a signal from a position sensor of a registration probe as the registration probe is positioned at each registration point of the first plurality of registration points. Each of the signals from the position sensor indicating a corresponding real-time position of the position sensor in three-dimensional space. Position data is captured for a second plurality of registration points along a lateral region of a head of a patient, based on a signal from a position sensor of a registration probe as the registration probe is positioned at each registration point of the second plurality of registration points. A real-time position of the patient is registered with an image guided surgery system, based on at least the captured position data for the first and second captured registration points.

This application claims priority under 35 U.S.C. § 119 to U.S. Patent Application Ser. No. 63/539,345, which was filed on Sep. 20, 2023 and is incorporated herein by reference in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the TRUDI® Navigation System by Acclarent, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

One function that may be performed by an IGS system is obtaining one or more reference points that may be used to correlate various preoperatively obtained images with a patient's actual position during a procedure. This act may be referred to as patient registration. Such registration may be performed by using a positionally tracked instrument (e.g., a registration probe whose tip position may be detected in three-dimensional space) to trace or touch one or more positions on a patient's face. At each touch point, the IGS system may register that point in three-dimensional space; and, using a number of registered points, determine the position of the affected area in three-dimensional space. Once the affected area is fully mapped or registered, it may be correlated with preoperative images in order to provide a seamless IGS experience across varying types of preoperative images during the performance of the procedure.

In some scenarios where a medical procedure is to be performed at a lateral side of a head of a patient (e.g., otology procedures, neurotology procedures, lateral skull base procedures, etc.), it may be desirable to perform the IGS system registration process at the lateral side of the head of the patient. Such registration may provide enhanced accuracy during subsequent IGS system navigation with sensor-equipped instruments that are inserted into the patient's head via the lateral side of the head of the patient. While several systems and methods have been made and used in connection with IGS navigation systems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 is a schematic view of an example of an IGS system, with a patient lying on their back;

FIG. 2 is a side schematic view of a patient and an example of a first registration plane;

FIG. 3 is a side schematic view of a patient and an example of a second registration plane;

FIG. 4 is a perspective view of an example of a registration probe;

FIG. 5 is a perspective view of the registration probe of FIG. 4 positioned for contacting several registration points on the first registration plane of FIG. 2, in the context of the IGS system of FIG. 1;

FIG. 6A is a perspective view of a patient with their head turned to a side in the context of the IGS system of FIG. 1, with a flap of skin peeled away to reveal bone;

FIG. 6B is a perspective view of the registration probe of FIG. 4 positioned for contacting several registration points on the second registration plane of FIG. 3, in the context of the IGS system of FIG. 1 with the bone revealed in FIG. 6A; and

FIG. 7 is a flow chart representing an example of a method of registering a patient with the IGS system of FIG. 1 using the registration plane of FIG. 2 and the registration plane of FIG. 3.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

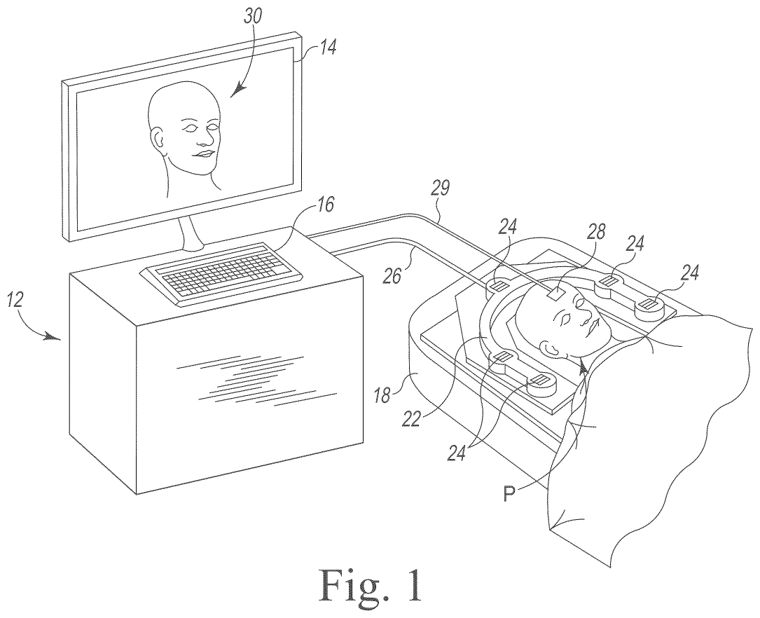

When performing a medical procedure within a head of a patient (P), it may be desirable to have information regarding the position of an instrument within the head of the patient (P), particularly when the instrument is in a location where it is difficult or impossible to obtain an endoscopic view of a working element of the instrument within the head of the patient (P). FIG. 1 shows an example of an IGS system 10 enabling a medical procedure to be performed within a head of a patient (P) using image guidance. In addition to or in lieu of having the components and operability described herein, the IGS navigation system 10 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 10,561,370, entitled “Apparatus to Secure Field Generating Device to Chair,” issued Feb. 18, 2020, the disclosure of which is incorporated by reference herein, in its entirety.

The IGS system 10 of the present example includes a field generator assembly 20, which includes a set of magnetic field generators 24 that are integrated into a horseshoe-shaped frame 22. The field generators 24 are operable to generate alternating magnetic fields of different frequencies around the head of the patient (P). In the present example, the frame 22 is positioned on a table 18, with the patient (P) lying on their back on the table 18 such that the frame 42 is located adjacent to the head of the patient (P).

The IGS system 10 of the present example further includes a processor 12, which controls the field generators 24 and other elements of the IGS system 10. For instance, the processor 12 is operable to drive the field generators 24 to generate alternating electromagnetic fields; and process signals from the instrument to determine the location of a navigation sensor or position sensor in the instrument within the head of the patient (P). The processor 12 includes a processing unit (e.g., a set of electronic circuits arranged to evaluate and execute software instructions using combinational logic circuitry or other similar circuitry) communicating with one or more memories. The processor 12 is coupled with the field generator assembly 20 via a cable 26 in this example, though the processor 12 may alternatively be coupled with the field generator assembly 20 wirelessly or in any other suitable fashion.

A display screen 14 and a user input feature 16 are also coupled with the processor 12 in this example. The user input feature 16 may include a keyboard, a mouse, a trackball, and/or any other suitable components, including combinations thereof. In some versions, the display screen 14 is in the form of a touchscreen that is operable to receive user inputs, such that the display screen 14 may effectively form at least part of the user input feature 160. A physician may use the input feature 16 to interact with the processor 12 while performing a registration process, while performing a medical procedure, and/or at other suitable times.

A medical instrument may include a navigation sensor or position sensor that is responsive to positioning within the alternating magnetic fields generated by the field generators 24. In some versions, the navigation sensor or position sensor of the instrument may comprise at least one coil at or near the distal end of the instrument. When such a coil is positioned within an alternating electromagnetic field generated by the field generators 24, the alternating magnetic field may generate electrical current in the coil, and this electrical current may be communicated as position-indicative signals via wire or wirelessly to the processor 12. This phenomenon may enable the IGS system 10 to determine the real-time location of the distal end of the instrument within a three-dimensional space (i.e., within the head of the patient (P), etc.). To accomplish this, the processor 12 executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

The processor 12 uses software stored in a memory of the processor 12 to calibrate and operate the IGS system 10. Such operation includes driving the field generators 24, processing data from the instrument, processing data from the user input feature 16, and driving the display screen 14. In some implementations, operation may also include monitoring and enforcement of one or more safety features or functions of the IGS system 10. The processor 12 is further operable to provide video and/or other images in real time via the display screen 14, showing the position of the distal end of the instrument in relation to a video camera image of the head of the patient (P), in relation to preoperative image (e.g., a CT scan image) of the head of the patient (P), and/or in relation to a computer-generated three-dimensional model of anatomical structures of the head of the patient (P). The display screen 14 may display such images simultaneously and/or superimposed on each other during the medical procedure. Such displayed images may also include graphical representations of instruments that are inserted in the head of the patient (P), or at least a position indicator (e.g., crosshairs, etc.), such that the operator may observe a visual indication of the instrument at its actual location in real time via the display screen 14.

In the example shown in FIG. 1, the display screen 14 is displaying a three-dimensional rendering 30 of the head of the patient (P). By way of further example only, the display screen 14 may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on the display screen 14. The images provided through the display screen 14 may thus help guide the operator in maneuvering and otherwise manipulating instruments within the head of the patient (P).

In the present example, the field generators 24 are in fixed positions relative to the head of the patient (P), such that the frame of reference for IGS system 10 (i.e., the electromagnetic field generated by the field generators 24) does not move with the head of the patient (P). In some instances, the head of the patient (P) may not remain completely stationary relative to the field generators 24 throughout the duration of a medical procedure, such that it may be desirable to track movement of the head of the patient (P) during a medical procedure. To that end, the IGS system 10 of the present example includes a tracking sensor 28 that is fixedly secured to the head of the patient (P). The tracking sensor 28 includes one or more coils and/or other position sensors that are operable to generate signals in response to the alternating magnetic fields generated by the field generators 24, with such signals indicating the position of the tracking sensor 28 in three-dimensional space. In the present example, these signals are communicated to the processor 12 via a cable 29. In some other versions, these signals are communicated to the processor 12 wirelessly.

Regardless of how the processor 12 receives signals from the tracking sensor 28, the processor 12 may utilize such signals to effectively track the real-time position of the head of the patient (P) and thereby account for any movement of the head of the patient (P) during a medical procedure. In other words, the processor 12 may process position-indicative signals from the tracking sensor 28 in combination with position-indicative signals from a position sensor-equipped medical instrument that is disposed in the head of a patient (P) to accurately determine the real-time position of the distal end (or other working feature) of the medical instrument in the head of the patient (P) despite any movement of the head of the patient (P) during the medical procedure.

In the example shown in FIG. 1, the tracking sensor 28 is positioned at the center pf the forehead of the patient (P), though it should be understood that the tracking sensor 28 may be positioned at any other suitable location on the head of the patient (P). By way of example only, the tracking sensor 28 may alternatively be positioned at the lateral forehead, at the upper orbital rim, in a lateral and/or posterior region of the head of the patient, or at some other location near the site at which the medical procedure will be performed in the head of the patient (P). In some variations, the tracking sensor 28 is positioned in the mouth of the patient (P). It should also be understood that the tracking sensor 28 may be fixedly secured to the head of the patient (P) in numerous ways, including but not limited to adhesives, screws, tacks, sutures, etc. In some cases, the tracking sensor 28 is secured to the skin of the head of the patient (P). In some other cases, the tracking sensor 28 is secured to the bone of the head of the patient (P).

### II. Example of Method of Registering a Patient to IGS System Along Two Registration Planes

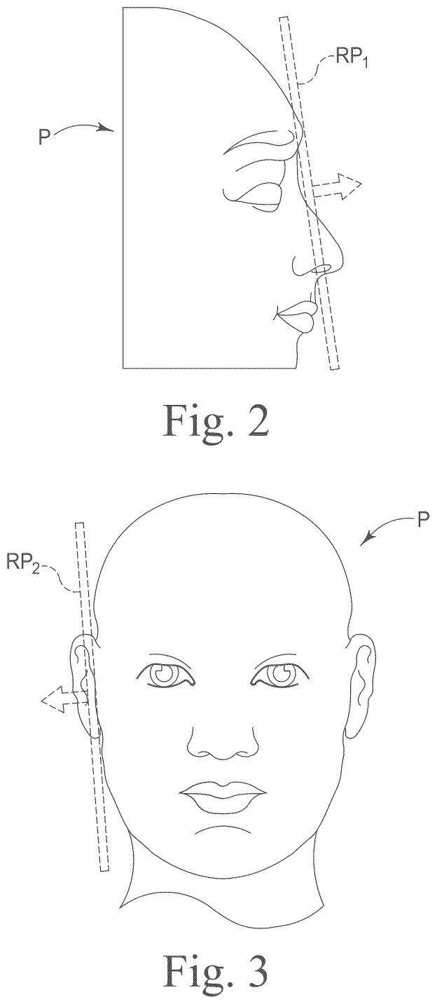

As noted above it may be necessary to register a patient (P) with the IGS system 10 in order to allow the processor 12 to initially correlate a real-time position of the patient (P) with one or more preoperative images (e.g., CT images, MRI images, three-dimensional model, etc.) of the patient (P), to thereby allow the processor 12 to track and visually indicate the real-time position of a position sensor-equipped instrument in the patient (P) via the display screen 14. Some conventional registration methods may tend to provide registration along only one registration plane, such as along only the front of a face of the patient (P). It may be advantageous to instead provide registration along at least two registration planes on the head of the patient (P). For instance, FIG. 2 shows a first registration plane (RP₁) positioned along the front of the face of the patient (P); while FIG. 2 shows a second registration plane (RP₂) positioned along the side of the head of the patient (P). The below description provides an example of how a patient (P) may be registered to an IGS system (10) along both of these registration planes (RP₁, RP₂).

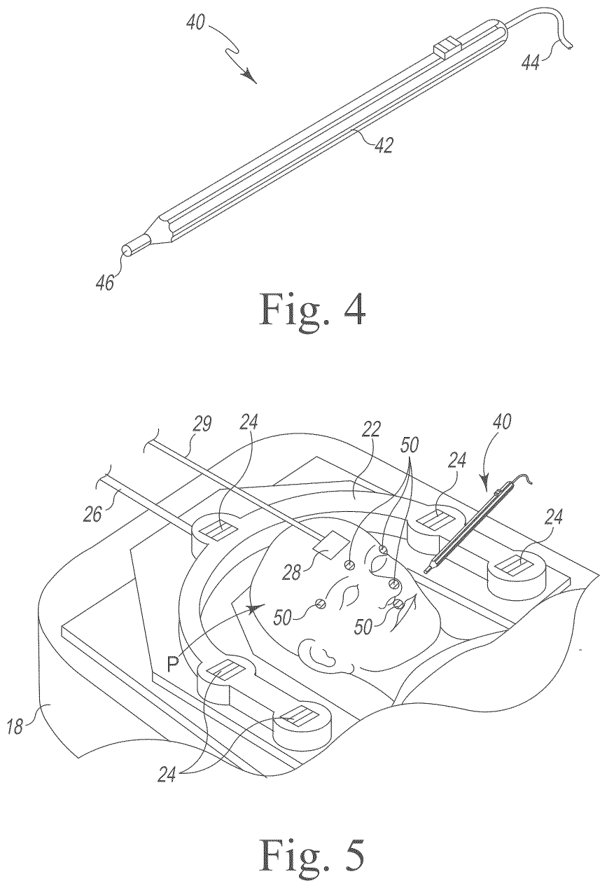

As also noted above, a registration process may be carried out using a registration probe. FIG. 4 shows an example of a registration probe 40. The registration probe 40 of this example includes a shaft 42 with a sensor-equipped distal tip 46. The shaft 42 is configured to be grasped by a hand of an operator during a registration process. The position sensor in the distal tip 46 may include one or more coils configured to generate signals in response to alternating electromagnetic fields generated by the field generators 24, with such signals indicating the real-time position of the distal tip 46 in three-dimensional space. A cable 44 extends proximally from the shaft 42 and provides a path for communicating position-indicative signals from the position sensor in the distal tip 46 to processor 12. In some other versions, the registration probe 40 is in wireless communication with the processor 12, such that the cable 44 may be omitted.

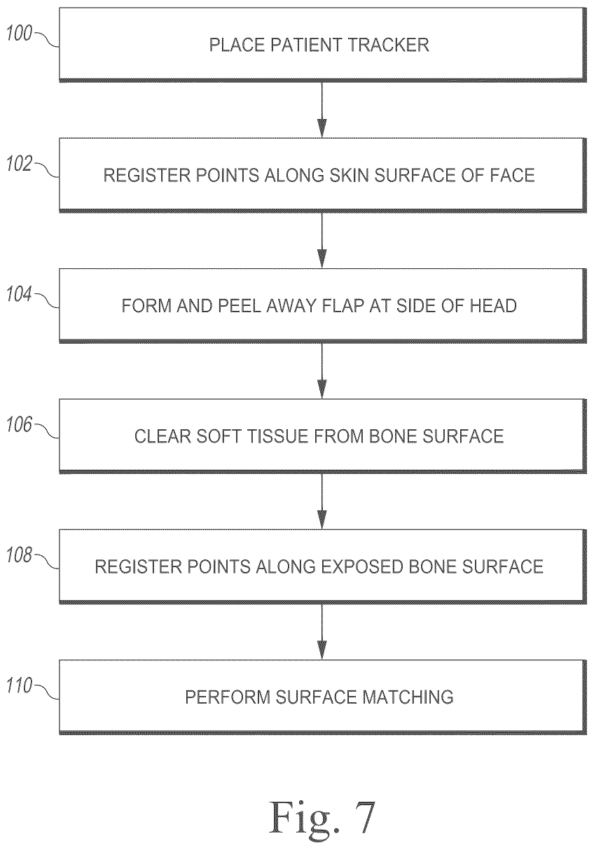

FIGS. 5-7 depict an example of a registration method that may be carried out to register a patient (P) with the IGS system 10 using two registration planes (RP₁, RP₂). The process may start with placement (block 100) of the tracking sensor 28 on the patient (P). By way of example only, the tracking sensor 28 may be placed on the front of the head of the patient (P) as shown in FIG. 1, on the side of the head of the patient (P), or elsewhere as described above. Next, the operator may utilize the registration probe 40 to register (block 102) several registration points along the skin surface of the face of the patient (P), thereby providing registration along the first registration plane (RP₁). FIG. 5 shows an example of this stage of the process, where several registration points 50 are positioned along the front of the face of the patient (P). As part of this stage of the process, the operator may gently touch the distal tip 46 to each of the registration points 50 on the skin of the patient, without pressing hard enough to deform the skin of the patient. Otherwise, if the operator presses hard enough to deform the skin of the patient with the distal tip 46, the registration at such registration points 50 may be unreliable.

The processor 12 may log the data from the position sensor at the distal tip 46 as each of these registration points 50 is registered. In some versions, the processor 12 may drive the display screen 14 to show the location of each registration point 50 on a three-dimensional rendering 30 of the head of the patient (P) (or otherwise convey the locations of registration points to the operator), to thereby guide the operator through this stage of the registration process. Similarly, the processor 12 may drive the display screen 14 to provide feedback to the operator indicating when each registration point 50 has been successfully registered.

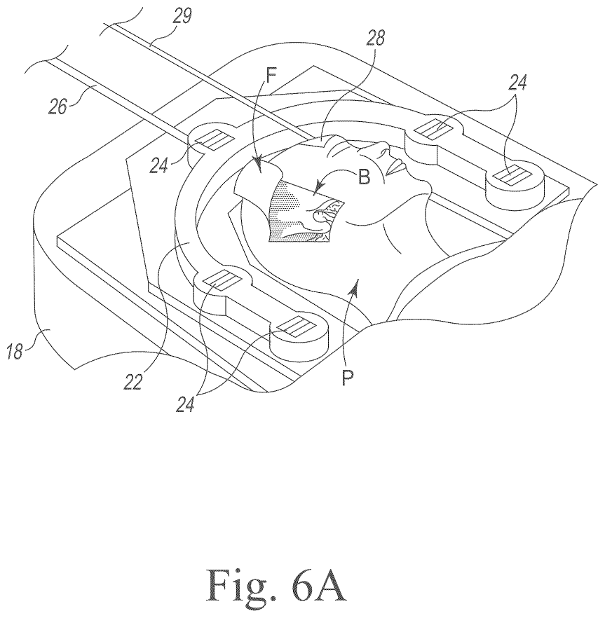

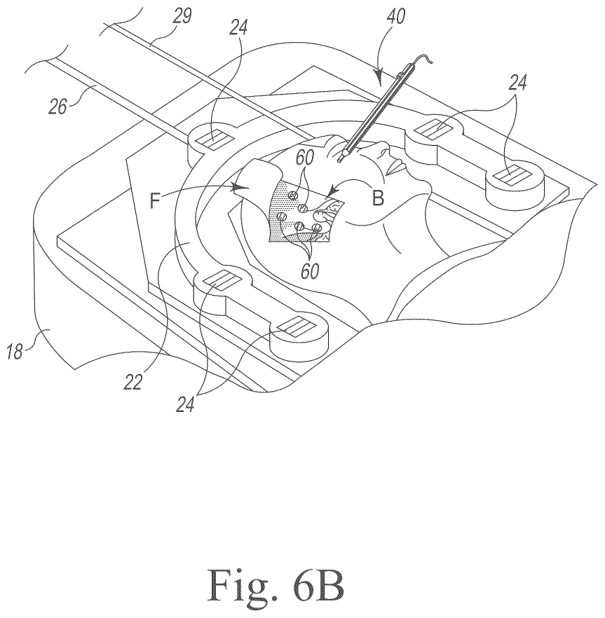

Once the desired number of registration points 50 have been registered (block 102) along the skin surface of the face of the patient (P), the operator may turn the head of the patient (P), then form and peel away (block 104) a flap of skin on the side of the head of the patient (P). An example of this is shown in FIG. 6A, where a flap (F) has been formed and peeled away to reveal bone (B) of the skull of the patient (P). In the present example, the flap (F) is formed in the periosteal region, such that the exposed bone (B) includes temporal bone. In some versions, the patient (P) remains lying on their back for this stage of the process, and only the head of the patient (P) is turned to the side. In some other versions, the entire body of the patient (P) is turned such that the patient (P) is lying on their side for this stage of the process. If any soft tissue remains on the bone (B) from which the flap (F) was peeled away, such soft tissue may be cleared (block 106) from the surface of the bone (B) such that only the bone (B) remains exposed from where the flap (F) was peeled away. Such full exposure of the bone (B) may avoid any inaccuracies in registration that might otherwise occur if the distal tip 46 were to contact soft tissue that remains on the bone (B) and deforms during registration. In other words, fully exposing the bone (B) from soft tissue may enhance the accuracy process by allowing the distal tip 46 to contact the exposed bone (B) directly, as the exposed bone (B) would not deform in response to pressing of the distal tip 46 against the contacted surface.

Once the bone (B) from which the flap (F) was peeled away is sufficiently exposed, the operator registers (block 108) points along the exposed bone surface, thereby providing registration along the second registration plane (RP₂). FIG. 6B shows an example of this stage of the process, where several registration points 60 are positioned along the exposed bone (B) of the patient (P). As part of this stage of the process, the operator may touch the distal tip 46 to each registration point 60 on the skin of the patient. Given the density of the bone (B), there may be little to no risk of the distal tip 46 causing any deformation of the bone (B), such that the registration at points 60 may be substantially reliable.

The processor 12 may log the data from the position sensor at the distal tip 46 as each of these registration points 60 is registered. In some versions, the processor 12 may drive the display screen 14 to show the location of each registration point 60 on a three-dimensional rendering 30 of the head of the patient (P) (or otherwise convey the locations of registration points to the operator), to thereby guide the operator through this stage of the registration process. Similarly, the processor 12 may drive the display screen 14 to provide feedback to the operator indicating when each registration point 60 has been successfully registered.

Once the desired number of registration points 60 have been registered (block 108), the processor 12 may perform surface matching with respect to the registration points 50 registered (block 102) along the skin surface of the face of the patient (P); and with respect to the registration points 60 registered (block 108) along the exposed bone (B) surface at the side of the head of the patient (P). This surface matching may correlate the registered points from both registration planes (RP₁, RP₂) with data from preoperative images (e.g., CT scans, MRI scans, etc.), thereby registering the real-time positions of various anatomical structures of the patient (P) with the same anatomical structures in the preoperative images. In other words, the registration data gathered from both sets of points 50 and 60 during both steps of registration (block 102, 108) may be merged to achieve registration with preoperative images with accuracy that may not otherwise be achieved in cases where registration is only carried out along one of the registration planes (RP₁, RP₂). With the enhanced registration complete, the processor 12 may subsequently drive the display screen 14 to superimpose a visual indication of the real-time position of a sensor-equipped instrument on the appropriate anatomical location in preoperative images (and/or on a digital model constructed based on data from preoperative images, etc.).

The process described above may be carried out in preparation for various kinds of medical procedures, including but not limited to ear, nose, and throat (ENT) procedures, cranial procedures, and neurotology procedures.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method, comprising: (a) capturing position data for a first plurality of registration points along an anterior region of a head of a patient, the position data for the first plurality of registration points being captured based on signals from a position sensor of a registration probe as the registration probe is positioned at each registration point of the first plurality of registration points, each of the signals from the position sensor indicating a corresponding real-time position of the position sensor in three-dimensional space; (b) capturing position data for a second plurality of registration points along a first lateral region of the head of the patient, the position data for the second plurality of registration points being captured based on signals from the position sensor of the registration probe as the registration probe is positioned at each registration point of the second plurality of registration points; and (c) registering a real-time position of the patient with an image guided surgery system, based on at least the captured position data for the first plurality of registration points and the captured position data for the second plurality of registration points, to thereby achieve registration of the patient with the image guided surgery system.

### Example 2

The method of Example 1, the head of the patient having a posterior region, the head of the patient being supported on the posterior region during the act of capturing position data for the first plurality of registration points.

### Example 3

The method of any of Examples 1 through 2, the head of the patient having a second lateral region opposite to the first lateral region, the head of the patient being supported on the second lateral region during the act of capturing position data for the second plurality of registration points.

### Example 4

The method of any of Examples 1 through 3, the anterior region of the head of the patient having skin, the act of capturing position data for the first plurality of registration points comprising contacting the skin with the registration probe at the first plurality of registration points.

### Example 5

The method of registration of any of Examples 1 through 4, the lateral region of the head of the patient having skin, the method further comprising: (a) forming a flap in the skin of the patient in the lateral region of the head of the patient; and (b) peeling the flap away from the head of the patient; the act of capturing position data for the first plurality of registration points comprising contacting the head of the patient in a region from which the flap was peeled away.

### Example 6

The method of Example 5, the act of forming the flap in the skin of the patient comprising forming the flap in a periosteal region of the head of the patient.

### Example 7

The method of any of Examples 5 through 6, the act of peeling the flap away from the head of the patient resulting in exposure of bone of the head of the patient.

### Example 8

The method of Example 7, the exposed bone comprising temporal bone.

### Example 9

The method of any of Examples 7 through 8, further comprising clearing additional soft tissue away from the bone.

### Example 10

The method of any of Examples 7 through 9, the act of capturing position data for the second plurality of registration points along the first lateral region of the head of the patient comprising contacting the bone with the registration probe.

### Example 11

The method of Example 1, further comprising: (a) driving a display to visually indicate to an operator locations of the first plurality of registration points along an anterior region of the head of the patient; and (b) driving the display to visually indicate to the operator locations of the second plurality of registration points along the first lateral region of the head of the patient.

### Example 12

The method of Example 11, the act of driving the display to visually indicate to the operator locations of the first plurality of registration points comprising rendering a first set of indicators on the display; the act of driving the display to visually indicate to the operator locations of the second plurality of registration points comprising rendering a second set of indicators on the display.

### Example 13

The method of Example 12, further comprising displaying a three-dimensional rendering of the head of the patient; the act of rendering the first set of indicators on the display comprising overlaying the first set of indicators on the three-dimensional rendering of the head of the patient; the act of rendering the second set of indicators on the display comprising overlaying the second set of indicators on the three-dimensional rendering of the head of the patient.

### Example 14

The method of any of Examples 1 through 13, further comprising receiving a patient tracking signal from a tracking sensor, the tracking sensor being fixedly secured to the head of the patient, the patient tracking signal indicating a real-time position of the head of the patient.

### Example 15

The method of Example 14, the act of registering the real-time position of the patient with the image guided surgery system being further based on the patient tracking signal.

### Example 16

The method of any of Examples 1 through 15, further comprising receiving a signal from a position sensor of a medical instrument, the medical instrument being disposed in the head of the patient, the signal from the position sensor of the medical instrument indicating a real-time position of the position sensor of the medical instrument in three-dimensional space.

### Example 17

The method of Example 16, the signal from the position sensor of the medical instrument indicating a real-time position of a distal end of the medical instrument in three-dimensional space.

### Example 18

The method of any of Examples 16 through 17, further comprising determining the real-time position of a portion of the medical instrument relative to the real-time position of the patient and further relative to a corresponding position in one or more preoperative images, based on at least the registration of the patient with the image guided surgery system and the signal from the position sensor of the medical instrument.

### Example 19

The method of Example 18, further comprising driving a display to render an indicator showing a real-time position of a portion of the medical instrument in relation to the one or more preoperative images.

### Example 20

The method of any of Examples 18 through 19, the one or more preoperative images comprising one or both of a CT scan image of the patient or a three-dimensional model of the patient.

### Example 21

The method of any of Examples 16 through 20, the medical instrument being inserted into the head of the patient via an ear of the patient on the first lateral side of the head of the patient.

### Example 22

A method, comprising: (a) capturing position data along a first registration plane associated with a head of a patient, the position data for the first registration plane being captured based on signals from a position sensor of a registration probe as the registration probe is positioned along the first registration plane, each of the signals from the position sensor indicating a corresponding real-time position of the position sensor in three-dimensional space; (b) capturing position data along a second registration plane associated with the head of the patient, the position data for the second registration plane being captured based on signals from the position sensor of the registration probe as the registration probe is positioned along the second registration plane; and (c) registering a real-time position of the patient with an image guided surgery system, based on at least the captured position data for the first registration plane and the captured position data for the second registration plane, to thereby achieve registration of the patient with the image guided surgery system.

### Example 23

The method of Example 22, the first registration plane being positioned along an anterior region of the head of the patient, the second registration plane being positioned along a lateral region of the head of the patient.

### Example 24

A method, comprising: (a) capturing position data for a first plurality of registration points along a skin surface of a head of a patient, the position data for the first plurality of registration points being captured based on signals from a position sensor of a registration probe as the registration probe is positioned at each registration point of the first plurality of registration points, each of the signals from the position sensor indicating a corresponding real-time position of the position sensor in three-dimensional space; (b) capturing position data for a second plurality of registration points along a bone surface of the head of the patient, the position data for the second plurality of registration points being captured based on signals from the position sensor of the registration probe as the registration probe is positioned at each registration point of the second plurality of registration points; and (c) registering a real-time position of the patient with an image guided surgery system, based on at least the captured position data for the first plurality of registration points and the captured position data for the second plurality of registration points, to thereby achieve registration of the patient with the image guided surgery system.

### Example 25

The method of Example 24, the skin surface being positioned along an anterior region of the head of the patient, the bone surface being positioned along a lateral region of the head of the patient.

### Example 26

The method of any of Examples 24 through 25, further comprising exposing the bone surface.

### Example 27

The method of Example 26, the act of exposing the bone surface comprising: (i) forming a flap in skin of the patient overlying the bone surface, and (ii) peeling the flap away from the bone surface.

### Example 28

The method of Example 27, further comprising clearing additional soft tissue away from the bone surface after the act of peeling the flap away from the bone surface.

### Example 29

The method of any of Examples 27 through 28, the flap comprising a periosteal flap.

### Example 30

The method of any of Examples 24 through 29, the skin surface being positioned on a forehead region of the patient, the bine surface being positioned along a temporal region of the patient.

### Example 31

A system comprising: (a) a field generating assembly operable to generate alternating magnetic fields around a head of a patient; (b) a registration probe including a position sensor operable to generate signals indicating a real-time position of a distal tip of the registration probe; and (c) a processor, the processor being configured to: (i) capture position data for a first plurality of registration points along an anterior region of a head of a patient, the position data for the first plurality of registration points being captured based on signals from a position sensor of a registration probe as the registration probe is positioned at each registration point of the first plurality of registration points, each of the signals from the position sensor indicating a corresponding real-time position of the position sensor in three-dimensional space, (ii) capture position data for a second plurality of registration points along a first lateral region of the head of the patient, the position data for the second plurality of registration points being captured based on signals from the position sensor of a registration probe as the registration probe is positioned at each registration point of the second plurality of registration points, and (iii) register a real-time position of the patient with an image guided surgery system, based on at least the captured position data for the first plurality of registration points and the captured position data for the second plurality of registration points, to thereby achieve registration of the patient with the image guided surgery system.

### Example 32

The system of Example 31, further comprising a display, the processor being further configured to drive the display to visually indicate information to an operator.

### Example 33

The system of Example 32, the processor being further configured to drive the display to visually indicate to an operator the first location and the second location.

### Example 34

The system of Example 33, the processor being further configured to display a three-dimensional rendering of the head of the patient and visually indicate the first and second locations on the three-dimensional rendering of the head of the patient.

### Example 35

The system of any of Examples 32 through 34, the processor being further configured to: (i) determine a real-time position of a medical instrument in relation to a patient, based at least in part on signals from a position sensor of the medical instrument, and (ii) drive the display to visually indicate to an operator a position of the medical instrument in relation to the one or more preoperative images.

### IV. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A method, comprising:
capturing position data for a first plurality of registration points along an anterior region of a head of a patient, the position data for the first plurality of registration points being captured based on signals from a position sensor of a registration probe as the registration probe is positioned at each registration point of the first plurality of registration points, each of the signals from the position sensor indicating a corresponding real-time position of the position sensor in three-dimensional space;
capturing position data for a second plurality of registration points along a first lateral region of the head of the patient, the position data for the second plurality of registration points being captured based on signals from the position sensor of the registration probe as the registration probe is positioned at each registration point of the second plurality of registration points; and
registering a real-time position of the patient with an image guided surgery system, based on at least the captured position data for the first plurality of registration points and the captured position data for the second plurality of registration points, to thereby achieve registration of the patient with the image guided surgery system.

2. The method of claim 1, wherein:
the head of the patient has a posterior region, and
the head of the patient is supported on the posterior region during capture of position data for the first plurality of registration points.

3. The method of claim 1, wherein:
the head of the patient has a second lateral region opposite to the first lateral region,
the head of the patient is supported on the second lateral region during capture of position data for the second plurality of registration points.

4. The method of claim 1, wherein:
the anterior region of the head of the patient has skin, and
capturing position data for the first plurality of registration points comprises contacting the skin with the registration probe at the first plurality of registration points.

5. The method of registration of claim 1, wherein the lateral region of the head of the patient has skin, the method further comprising:
forming a flap in the skin of the patient in the lateral region of the head of the patient; and
peeling the flap away from the head of the patient;
wherein capturing position data for the first plurality of registration points comprises contacting the head of the patient in a region from which the flap was peeled away.

6. The method of claim 5, wherein forming the flap in the skin of the patient comprises forming the flap in a periosteal region of the head of the patient.

7. The method of claim 5, peeling the flap away from the head of the patient results in exposure of bone of the head of the patient.

8. The method of claim 7, wherein the exposed bone comprises temporal bone.

9. The method of claim 7, further comprising clearing additional soft tissue away from the bone.

10. The method of claim, wherein capturing position data for the second plurality of registration points along the first lateral region of the head of the patient comprises contacting the bone with the registration probe.

11. The method of claim 1, further comprising:
driving a display to visually indicate to an operator locations of the first plurality of registration points along an anterior region of the head of the patient; and
driving the display to visually indicate to the operator locations of the second plurality of registration points along the first lateral region of the head of the patient.

12. The method of claim 11, wherein driving the display to visually indicate to the operator locations of the first plurality of registration points comprises rendering a first set of indicators on the display; and
driving the display to visually indicate to the operator locations of the second plurality of registration points comprises rendering a second set of indicators on the display.

13. The method of claim 12, further comprising displaying a three-dimensional rendering of the head of the patient, wherein:
rendering the first set of indicators on the display comprises overlaying the first set of indicators on the three-dimensional rendering of the head of the patient; and
rendering the second set of indicators on the display comprises overlaying the second set of indicators on the three-dimensional rendering of the head of the patient.

14. The method of claim 1, further comprising receiving a patient tracking signal from a tracking sensor, wherein:
the tracking sensor is fixedly secured to the head of the patient, and
the patient tracking signal indicates a real-time position of the head of the patient.

15. The method of claim 14, registering the real-time position of the patient with the image guided surgery system is further based on the patient tracking signal.

16. The method of claim 1, further comprising receiving a signal from a position sensor of a medical instrument, wherein:
the medical instrument is disposed in the head of the patient, and
the signal from the position sensor of the medical instrument indicates a real-time position of the position sensor of the medical instrument in three-dimensional space.

17. The method of claim 16, wherein the signal from the position sensor of the medical instrument indicates a real-time position of a distal end of the medical instrument in three-dimensional space.

18. The method of claim 16, further comprising determining the real-time position of a portion of the medical instrument relative to the real-time position of the patient and further relative to a corresponding position in one or more preoperative images, based on at least the registration of the patient with the image guided surgery system and the signal from the position sensor of the medical instrument.

19. The method of claim 18, further comprising driving a display to render an indicator showing a real-time position of a portion of the medical instrument in relation to the one or more preoperative images.

20. The method of claim 18, the one or more preoperative images comprising one or both of a CT scan image of the patient or a three-dimensional model of the patient.

21. The method of claim 16, the medical instrument being inserted into the head of the patient via an ear of the patient on the first lateral side of the head of the patient.
